(19) United States
(12) Patent Application Publication
(10) Pub. No.: US 2019/0117508 A1
(43) Pub. Date: Apr. 25, 2019

(54) BENEFICIAL BODY INSTRUMENT

(71) Applicant: Avex Group Holdings Inc., Tokyo (JP)

(72) Inventor: YASUKO KIMURA, (US)

(73) Assignee: Avex Group Holdings Inc., Tokyo (JP)

(21) Appl. No.: 15/559,701

(22) PCT Filed: Dec. 7, 2016
(86) PCT No.: PCT/JP2016/086339

§ 371 Date: Sep. 19, 2017

(51) Int. Cl. A61H 39/00; A61N 2/00; H04R 1/10

(52) U.S. Cl. CPC A61H 39/00; A61N 2/004; A61H 2205/027; H04R 1/105; A61H 2201/165; H04R 1/1016

(57) ABSTRACT

A beneficial body instrument includes a hook portion wrapped around an auricle to be detachably worn on the auricle, and magnetic stimulation portions provided at the hook portion to stimulate acupuncture points around the auricle.

### TECHNICAL FIELD

The present invention relates to a beneficial body instrument.

### BACKGROUND ART

It is known that beneficial effects such as lift-up due to blood flow improvement are obtained by stimulating, acupuncture points (acupoints) existing in various parts of the human body. Nowadays, various beneficial body instruments to self-stimulate acupuncture points to easily obtain the beneficial effects described above are available.

An earphone that includes an acupoint-stimulating material (a magnet or the like) is disclosed in Patent Literature 1. According to this constitution, acupuncture points in an external auditory meatus are stimulated due to the acupoint-stimulating material just by wearing the earphone in the external auditory meatus. In this way, by stimulating acupuncture points while listening to music during walking, running, doing housework, and the like, so-called “ beauty while doing something” is possible.

### CITATION LIST

### Patent Literature

### Patent Literature 1

Japanese Utility Model Registration No. 3113803

### SUMMARY OF INVENTION

### Technical Problem

However, in Patent Literature 1, the acupoint-stimulating material is provided in the earphone itself. Therefore, the conventional acupoint-stimulating material can only stimulate acupuncture points in the external auditory meatus, and it is difficult to stimulate acupuncture points around an auricle.

Acupuncture points cannot be stimulated when the earphone is not used, and an opportunity or time to stimulate acupuncture points is limited.

Accordingly, the present invention has been devised in consideration of the above circumstances, and an objective thereof is to provide a beneficial body instrument capable of easily obtaining beneficial effects by stimulating acupuncture points around an auricle.

### Solution to Problem

In the beneficial body instrument according to an aspect of the present invention, the following constitution was adopted to solve the above-mentioned problem.

(1) According to one aspect of the present invention, a beneficial body instrument is provided, including a hook portion detachably worn on an auricle to be wrapped around the auricle, and magnetic stimulation portions provided at the hook portion to stimulate acupuncture points around the auricle.

According to the above aspect (1), in a state in which the hook portion is worn on the auricle, magnetic fields generated from the magnetic stimulation portions act on acupuncture points around the auricle, and the acupuncture points are stimulated due to magnetism or the like. In this way, beneficial effects such as lift-up due to blood flow promotion can be obtained.

Particularly, because acupuncture points can be stimulated just by wearing the hook portion in the auricle, beauty while doing something can be easily achieved. Unlike the conventional constitution in which the acupoint-stimulating material is provided in the earphone itself, beneficial effects can be obtained regardless of opportunity or time.

(2) In the beneficial body instrument according to the above aspect (1), the magnetic stimulation portions may include a first magnetic stimulation portion provided at a portion of the hook portion located in front of the auricle to stimulate the Gall Bladder 2 (GB-2) acupuncture point, and a second magnetic stimulation portion provided at a portion of the hook portion located behind the auricle to stimulate the Gall Bladder 12 (GB-12) acupuncture point.

According to the above aspect (2), because the first magnetic stimulation portion that stimulates the GB-2 point and the second magnetic stimulation portion that stimulates the GB-12 point are provided, the GB-2 point and the GB-12 point can be simultaneously stimulated due to magnetism. As a result, beneficial effects can be more remarkably obtained.

(3) In the beneficial body instrument according to the above aspect (1) or (2), at the portions of the hook portion at which the magnetic stimulation portions are provided, protrusions protruding toward a user in a state in which the hook portion is worn on the auricle may be provided.

According to this constitution, because the magnetic stimulation portions can be brought closer to the user, beneficial effects due to magnetism can be further enhanced. Because the acupuncture points can be pressed by the protrusions, an acupressure effect may be given to the acupuncture points, and further beneficial effects can be obtained.

(4) In the beneficial body instrument according to any one of the above aspects (1) to (3), the hook portion may be configured to be deformable.

According to this constitution, because the hook portion is configured to be deformable, a fitting feeling can be improved by deforming a shape of the hook portion according to a shape of the auricle. By deforming the hook portion according to locations of the acupuncture points, the acupuncture points can be effectively stimulated.

(5) In the beneficial body instrument according to any one of the above aspects (1) to (4), an earphone-holding portion configured to detachably hold an earphone may be provided at the hook portion.

According to this constitution, because the earphone-holding portion configured to hold the earphone is provided, the acupuncture points can be stimulated while listening to music or the like. Because the earphone is detachable from the earphone-holding portion, the beneficial body instrument can be used with the earphone removed when the earphone is not used. Therefore, beneficial effects can be obtained regardless of opportunity or time.

### Advantageous Effects of Invention

According to the present invention, beneficial effects can be easily obtained by stimulating acupuncture points around an auricle.

### BRIEF DESCRIPTION OF DRAWINGS

FIG. 1 is a side view illustrating a state in which an earphone hook is worn on an auricle according to a first embodiment of the present invention.

FIG. 2 is a perspective view of the earphone hook illustrated in FIG. 1 viewed from the front.

FIG. 3 is a perspective view of the earphone hook illustrated in FIG. 1 viewed from the rear.

FIG. 4 is a side view of the earphone hook illustrated in FIG. 1.

FIG. 5 is a view illustrating acupuncture points located around the auricle.

FIG. 6 is a perspective view of an earphone hook according to a second embodiment of the present invention viewed from the rear.

FIG. 7 is a side view of the earphone hook illustrated in FIG. 6.

FIG. 8 is a side view illustrating a state in which the earphone hook illustrated in FIG. 6 is worn on an auricle in a first wearing mode.

FIG. 9 is a side view illustrating a state in which the earphone hook illustrated in FIG. 6 is worn on the auricle in a second wearing mode.

FIG. 10 is a side view of an earphone hook according to a third embodiment of the present invention.

FIG. 11 is a perspective view of the earphone hook illustrated in FIG. 10 viewed from the rear.

FIG. 12 is a side view illustrating a state in which the earphone hook illustrated in FIG. 10 is worn on an auricle in a first wearing mode.

FIG. 13 is a side view illustrating a state in which the earphone hook illustrated in FIG. 10 is worn on the auricle in a second wearing mode.

### DESCRIPTION OF EMBODIMENTS

Next, embodiments of the present invention will be described with reference to the drawings.

### (First Embodiment)

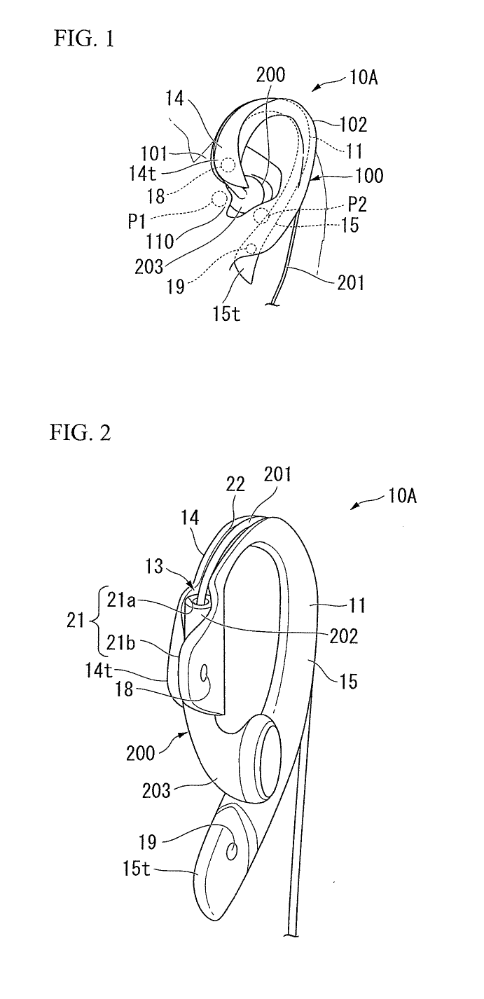

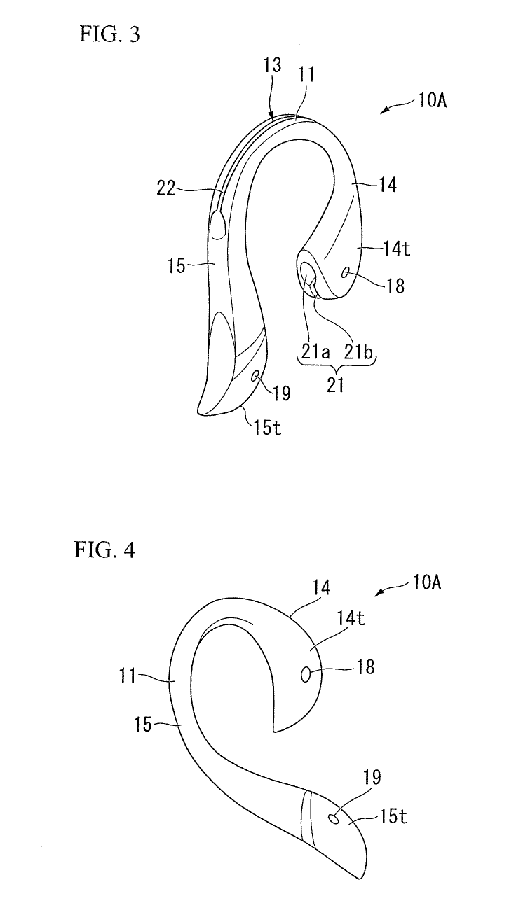

FIG. 1 is a side view illustrating a state in which an earphone hook WA is wont on an auricle according to a first embodiment. FIG. 2 is a perspective view of the earphone hook 10A viewed from the front. FIG. 3 is a perspective view of the earphone hook 10A viewed from the rear. FIG. 4 is a side view of the earphone hook 10A.

As illustrated in FIGS. 1 to 4, the earphone hook (beneficial body instrument) 10A stimulates acupuncture points located around an auricle 100 while holding an earphone 200 in the auricle 100. Directions such as a front-rear direction, a top-bottom direction, and a left-right direction described below are directions in a state in which the earphone hook 10A is worn by a user unless particularly mentioned otherwise. A pair of earphone hooks 10A according to the present embodiment are worn on each auricle 100 at both left and right sides of the user. However, because each of the earphone hooks 10A has the same constitution, the earphone hook 10A worn on the auricle 100 at one side (the left side of the user) will be mainly described in the following description.

As illustrated in FIG. 2, the earphone 200 includes a columnar cable-holding portion 202 configured to support a distal end portion of a cable 201, and a speaker housing 203 enlarged with respect to the cable-holding portion 202 and having a built-in speaker (not illustrated).

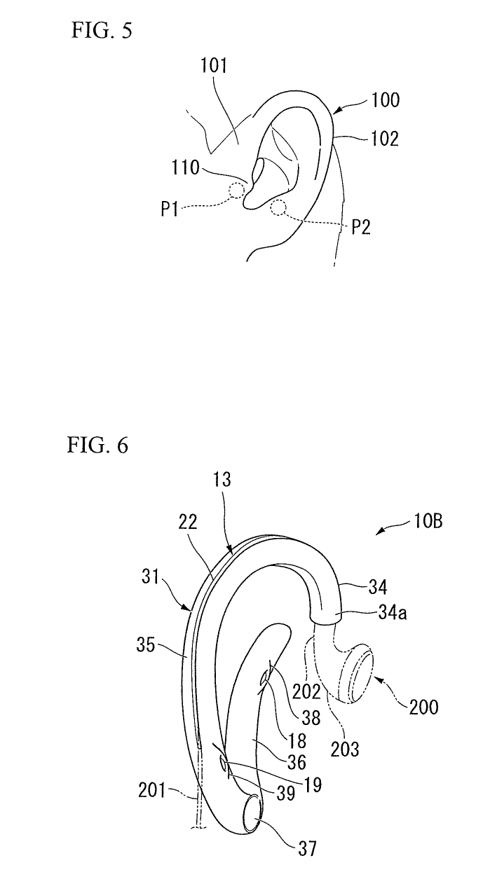

FIG. 5 is a view illustrating acupuncture points located around the auricle 100.

The earphone hook 10A according to the present embodiment mostly stimulates the Gall Bladder 2 (GB-2) point P1 and the Gall Bladder 12 (GB-12) point P2 among the acupuncture points located around the auricle 100 illustrated in FIG. 5. The GB-2 point Pt is an acupuncture point located in front of a tragus 110. The GB-12 point P2 is an acupuncture point located behind the auricle 100 (near a mastoid process).

As illustrated in FIGS. 1 to 4, the earphone hook 10A includes a hook portion 11, magnetic stimulation portions 18 and 19, and an earphone-holding portion 13. The hook portion 11 is curved in a J-shape as a whole. The hook portion 11 is detachably worn on the auricle 100 by being wrapped around the auricle 100 of the user (see FIG. 1). Specifically, the hook portion 11 includes a front side support 14 configured to hold the auricle 100 from the front and a rear side support 15 configured to hold the auricle 100 from the rear in a state in which the hook portion 11 is worn on the auricle 100.

In a side view in the left-right direction, the front side support 14 is formed in an arc shape protruding forward. The front side support 14 is wrapped around a front of the auricle 100 and extends in the top-bottom direction on a head surface 101 in the front of the auricle 100 (see FIG. 1). A lower end portion of the front side support 14 is arranged at a position that is, for example, in the vicinity of or in contact with the above-described GB-2 point P1. A front side tab portion 14t bulging forward is formed at the lower end portion of the front side support 14. The front side tab portion 14t is formed in a semicircular shape in the side view that is in the view in the left-right direction.

As illustrated in FIGS. 1 to 4, the rear side support 15 is formed in an arc shape protruding rearward. The rear side support 15 extends rearward from an upper end portion of the front side support 14 and then extends downward. In this way, the rear side support 15 passes through an inside of an earlobe 102 (between the head surface 101 and the earlobe 102 in the left-right direction) and is wrapped rearward around the auricle 100 from an upper portion thereof. A lower end portion of the rear side support 15 is arranged at a position that is, for example, in the vicinity of or in contact with the above-described GB-12 point P2. A rear side tab portion 15t bulging forward is formed at the lower end portion of the rear side support 15. The rear side tab portion 15t is formed in a semicircular shape in the side view that is in the view in the left-right direction. The rear side tab portion 15t is in the vicinity of or in contact with the auricle 100 inside the earlobe 102 from the rear thereof.

The hook portion 11 is formed by molding a core material (for example, a metal wire or the like) to a covering material (for example, a resin material, a rubber material, or the like). Therefore, the hook portion 11 is configured to be deformable according to a size or shape of the auricle 100 of the user. The hook portion 11 may be formed so that, for example, an area between the front side tab portion 14t and the rear side tab portion 15t is deformable, and the front side tab portion 14t and the rear side tab portion 15t are formed of a material that is harder than a material of the area between the front side tab portion 14t and the rear side tab portion 15t.

The magnetic stimulation portions 18 and 19 include a first magnetic stimulation portion 18 provided at the front side support 14 and a second magnetic stimulation portion 19 provided at the rear side support 15. In the present embodiment, each of the magnetic stimulation portions 18 and 19 is formed with a magnetic material containing 29 to 32% of Nd, 0.8 to 1.2% of Dy, 0.2 to 0.4% of Al, 1.0 to 1.2% of B, 64 to 69% of Fe, 0.5 to 1.0% of Nb, and the like. Each of the magnetic stimulation portions 18 and 19 preferably has a magnetic flux density of, for example, about 50 mT.

The first magnetic stimulation portion 18 is embedded in the front side tab portion 14t of the front side support 14. At least a portion of the first magnetic stimulation portion 18 is exposed on a surface of the front side tab portion 14t facing the head surface 101 (an inner surface of the front side tab portion 14t). The first magnetic stimulation portion 18 forms a magnetic field around the GB-2 point P1 and stimulates the GB-2 point P1 due to magnetism.

The second magnetic stimulation portion 19 is embedded in the rear side tab portion 15t of the rear side support 15. At least a portion of the second magnetic stimulation portion 19 is exposed on a surface of the rear side tab portion 15t facing the head surface 101 (an inner surface of the rear side tab portion 15t). The second magnetic stimulation portion 19 forms a magnetic field around the GB-12 point P2 and stimulates the GB-12 point P2 due to magnetism. The magnetic stimulation portions 18 and 19 may be entirely embedded in the supports 14 and 15 as long as the magnetic stimulation portions 18 and 19 have magnetic forces that can sufficiently stimulate the GB-2 point P1 and the GB-12 point P2.

In the same earphone hook 10A, each of the magnetic stimulation portions 18 and 19 has the same magnetic pole facing inward in the left-right direction. Between a pair of left and right earphone hooks 10A, magnetic poles facing inward in the left-right direction are different between fellow the first magnetic stimulation portions 18 and between fellow the second magnetic stimulation portions 19. In this way, magnetic force lines extending from the magnetic stimulation portions 18 and 19 form a closed loop between the pair of left and right earphone hooks 10A.

As illustrated in FIGS. 2 and 3, the earphone-holding portion 13 includes an insertion portion 21 and a cable guide groove 22.

The insertion portion 21 is formed at a lower portion of the front side support 14. The insertion portion 21 is formed in the shape of a groove extending in the top-bottom direction. Specifically, the insertion portion 21 includes a holding hole 21a into which the cable-holding portion 202 is detachably fitted and a slit 21b that allows communication between the holding hole 21a and an outer peripheral surface of the front side support 14.

The cable-holding portion 202 is fitted into the holding hole 21a from a lower side or front side (from the slit 22b side) thereof while the speaker housing 203 faces downward.

The cable guide groove 22 is formed to be continuous in an extending direction of the hook portion 11 across a range from an upper portion of the front side support 14 to an upper portion of the rear side support 15. The cable guide groove 22 communicates with an inside of the insertion portion 21 (the holding hole 21a) at a front end portion thereof. The cable guide groove 22 accommodates the cable 201 withdrawn from the cable-holding portion 202 and arranges the cable 201 from the front to the rear of the auricle 100 through the upper portion of the auricle 100.

When the above-described earphone hook 10A is used, for example, the earphone 200 is first worn on the earphone-holding portion 13. Specifically, the cable-holding portion 202 is fitted into the holding hole 21a from the lower side or front side thereof in a state in which the speaker housing 203 faces downward. Then, the cable 201 withdrawn from the cable-holding portion 202 is accommodated in the cable guide groove 22.

Next, the hook portion 11 of the earphone hook 10A is wrapped rearward from a front portion of the auricle 100 of the user to be worn. Here, the hook portion 11 is worn on the auricle 100 while the front side support 14 and the rear side support 15 are grasped and the hook portion 11 is deformed according to the shape of the auricle 100. Then, the speaker housing 203 is set in an external auditory meatus. In this way, the earphone 200 is worn on the auricle 100 using the earphone hook 10A.

Here, the front side support 14 of the hook portion 11 is arranged at a portion of the head surface 101 located in front of the auricle 100 in a state in which the earphone hook 10A is worn. The rear side support 15 is arranged at a portion located behind the auricle 100. In this way, the first magnetic stimulation portion 18 is arranged at a position in the vicinity of or in contact with the GB-2 point P1, and the second magnetic stimulation portion 19 is arranged at a position in the vicinity of or in contact with the GB-12 point P2.

Then, the magnetic field generated by the first magnetic stimulation portion 18 acts on the GB-2 point P1, and the magnetic field generated by the second magnetic stimulation portion 19 acts on the GB-12 point P2 such that the GB-2 point P1 and the GB-12 point P2 are stimulated due to magnetism or the like. In this way, beneficial effects such as lift-up due to blood flow promotion can be obtained.

Particularly, in the present embodiment, because the GB-2 point P1 and the GB-12 point. P2 can be stimulated just by wearing the hook portion 11 in the auricle 100, beauty while doing something can be easily achieved.

Further, unlike the conventional constitution in which the acupoint-stimulating material is provided in the earphone itself, beneficial effects can be obtained regardless of opportunity or time.

In the present embodiment, because the first magnetic stimulation portion 18 that stimulates the GB-2 point P1 and the second magnetic stimulation portion 19 that stimulates the GB-12 point P2 are provided, the GB-2 point P1 and the GB-12 point P2 can be simultaneously stimulated due to magnetism, and beneficial effects can be more remarkably obtained.

Because the earphone hook 10A according to the present embodiment includes the earphone-holding portion 13 that holds the earphone 200, acupuncture points around the auricle 100 can be stimulated while listening to music or the like. Because the earphone 200 is detachable, the earphone hook 10A can be used with the earphone 200 removed when the earphone 200 is not used. Therefore, beneficial effects can be obtained regardless of opportunity or time.

Further, in the present embodiment, because the hook portion 11 is configured to be deformable, a fitting feeling can be improved by deforming the shape of the hook portion 11 according to the shape of the auricle 100. The GB-2 point P1 and the GB-12 point P2 can be effectively stimulated by deforming the hook portion 11 according to locations of the GB-2 point P1 and the GB-12 point P2.

### (Second Embodiment)

Next, a beneficial body instrument according to a second embodiment of the present invention will be described.

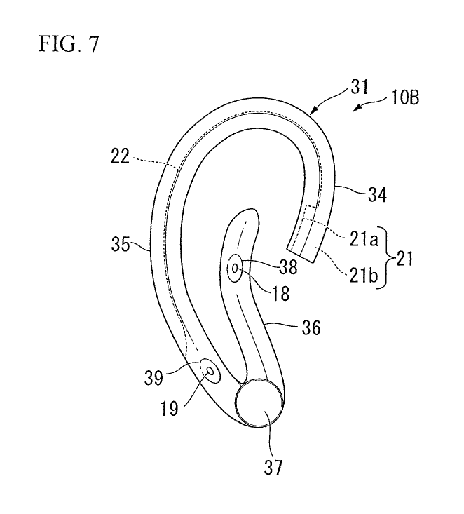

FIG. 6 is a perspective view of an earphone hook 10B according to the second embodiment viewed from the rear. FIG. 7 is a side view of the earphone hook 10B. In the following description, like reference numerals are given to the same constitutions as in the above-described first embodiment, and descriptions thereof may be omitted in some cases.

As illustrated in FIGS. 6 and 7, the earphone hook (beneficial body instrument) 10B includes a hook portion 31, the magnetic stimulation portions 18 and 19, and the earphone-holding portion 13.

The hook portion 31 includes a front side support 34, a rear side support 35, and a support arm 36 extending upward from a lower end portion of the rear side support 35.

A joint portion 37 is provided at the lower end portion of the rear side support 35. The joint portion 37 is formed of a material that is harder than materials of the supports 34 and 35 and the support arm 36.

The support arm 36 extends upward from the joint portion 37. Like the supports 34 and 35, the support arm 36 is formed by molding a core material (a metal wire or the like) to a covering material (a resin material, a rubber material, or the like) and is configured to be deformable according to the shape of the auricle 100 of the user. In this case, the support arm 36 is deformed around the joint portion 37 so that, for example, the support arm 36 approaches or recedes from the rear side support 35 in the front-rear direction.

The first magnetic stimulation portion 18 is embedded in an upper portion of die support arm 36. The second magnetic stimulation portion 19 is embedded in the lower end portion of the rear side support 35. Protrusions 38 and 39 protruding inward (toward the user) in the left-right direction are respectively provided at portions of the hook portion 11 (the support arm 36 and the rear side support 35) at which the magnetic stimulation portions 18 and 19 are embedded. The protrusions 38 and 39 are formed, for example, in a truncated cone shape. The magnetic stimulation portions 18 and 19 are respectively exposed at top portions of the protrusions 38 and 39.

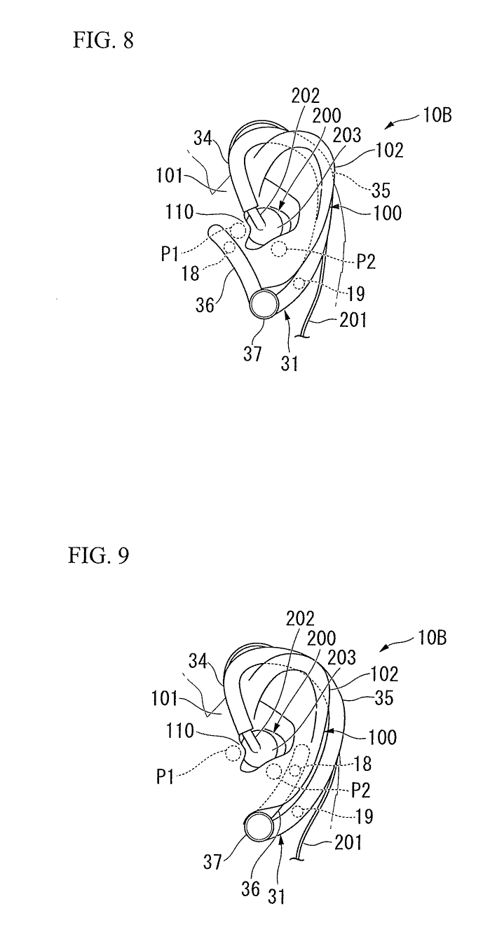

FIG. 8 is a side view illustrating a state in which the earphone hook 1013 is worn on the auricle 100 in a first wearing mode. FIG. 9 is a side view illustrating a state in which the earphone hook 10B is worn on the auricle 100 in a second wearing mode.

The earphone hook 10B according to the present embodiment can be worn on the auricle 100 with any one of the first wearing mode illustrated in FIG. 8 and the second wearing mode illustrated in FIG. 9.

When the earphone hook 10B is worn with the first wearing mode illustrated in FIG. 8, the hook portion 31 is worn on the auricle 100 embodiment in a state in which the support arm 36 is deformed forward with respect to the auricle 100, as in the above-described first. Then, the support arm 36 is deformed rearward. In this way, the auricle 100 is sandwiched between the front side support 34 the rear side support 35, and sandwiched between the support arm 36 and the rear side support 35. In this case, the first magnetic stimulation portion 18 of the support arm 36 is in the vicinity of or in contact with the GB-2 point P1. The second magnetic stimulation portion 19 of the rear side support 35 is in the vicinity of or in contact with the GB-12 point P2.

When the earphone hook 10B is worn with the second wearing mode illustrated in FIG. 9, the hook portion 31 is worn on the auricle 100 in a state in which the support arm 36 is deformed rearward with respect to the auricle 100, as in the above-described first embodiment. Here, the support arm 36 is arranged between the rear side support 35 and the auricle 100. In this way, the hook portion 31 is worn on the auricle 100 in a state in which a biasing force (a restoring force) for biasing the auricle 100 forward acts on the support arm 36. In this case, both the first magnetic stimulation portion 18 and the second magnetic stimulation portion 19 are arranged in the vicinity of the GB-12 point P2.

According to the present embodiment, because the auricle 100 can be held by the support arm 36, the earphone hook 10B can be more firmly worn on the auricle 100 in addition to having the same effect as the above-described first embodiment. In this way, misalignment of the earphone hook 10B with respect to the auricle 100 can be suppressed.

Because the protrusions 38 and 39 are respectively provided at portions of the hook portion 31 in which the magnetic stimulation portions 18 and 19 are embedded and the magnetic stimulation portions 18 and 19 can be brought closer to the user, beneficial effects due to magnetism can be further enhanced. Because the GB-2 point P1 and the GB-12 point P2 can be pressed by the protrusions 38 and 39, an acupressure effect can be given to the GB-2 point P1 and the GB-12 point P2, and further beneficial effects can be obtained.

### (Third Embodiment)

Next, a beneficial body instrument according to a third embodiment of the present invention will be described.

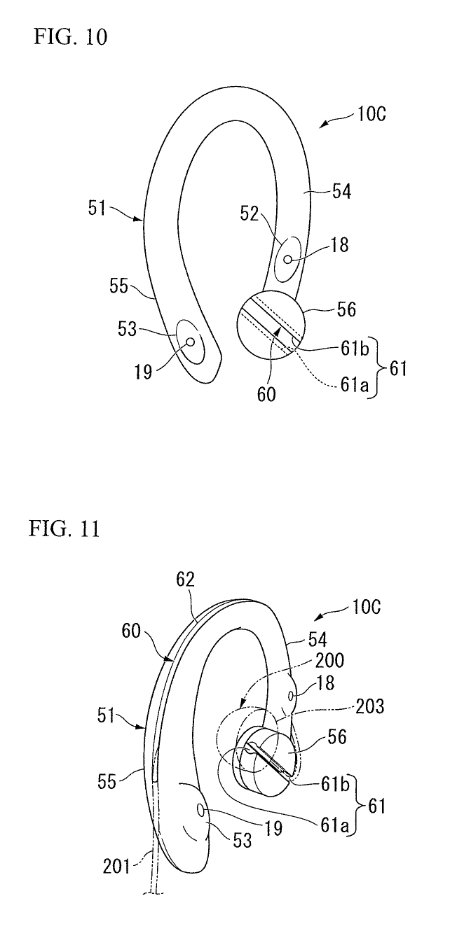

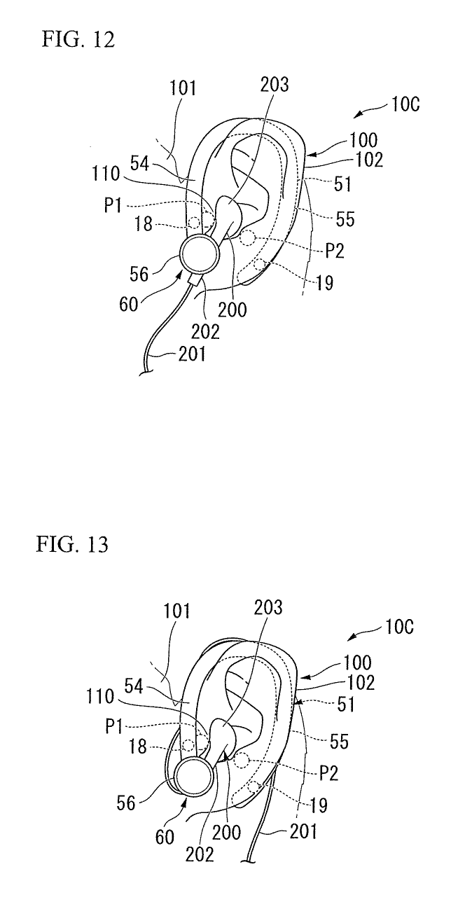

FIG. 10 is a side view of an earphone hook 10C according to the third embodiment. FIG. 11 is a perspective view of the earphone hook 100 viewed from the rear according to the third embodiment. FIG. 12 is a side view illustrating a state in which the earphone hook 10C is worn on the auricle 100 in a first wearing mode. FIG. 13 is a side view illustrating a state in which the earphone hook 10C is worn on the auricle 100 in a second wearing mode. In the following description, like reference numerals are given to the same constitutions as in the above-described embodiments, and descriptions thereof may be omitted in some cases.

In the earphone hook (beneficial body instrument) IOC illustrated in FIGS. 10 and 11, a hook portion 51 includes a front side support 54 and a rear side support 55. As illustrated in FIGS. 12 and 13, a lower end portion of the front side support 54 is located below the tragus 110 in a state in which the earphone hook 100 is worn by the user. A protrusion 52 protruding inward (toward the user) in the left-right direction is provided at the lower end portion of the front side support 54. The first magnetic stimulation portion 18 is embedded in the protrusion 52. A base portion 56 formed of a material that is harder than that of the hook portion 51 is provided at the lower end portion of the front side support 54.

A protrusion 53 protruding inward in the left-right direction is provided at a lower end portion of the rear side support 55. The second magnetic stimulation portion 19 is embedded in the protrusion 53.

As illustrated in FIG. 11, an insertion portion 61 of an earphone-holding portion 60 includes a holding hole 61a passing through the above-described base portion 56 and a slit 61b that allows communication between the holding hole 61a and an inside of the base portion 56.

The holding hole 61a gradually extends upward toward the rear. The cable-holding portion 202 of the earphone 200 is inserted into the holding hole 61a, for example, obliquely from a rear upper side thereof.

A cable guide groove 62 of the earphone-holding portion 60 is formed in an extending direction of the hook portion 51 at an outer peripheral portion of the hook portion 51. Specifically, the cable guide groove 62 is formed across a range from the lower end portion of the front side support 54 to an intermediate portion of the rear side support 55 in the top-bottom direction.

The earphone hook IOC according to the present embodiment can be worn on the auricle 100 with any one of the first wearing mode illustrated in FIG. 12 and the second wearing mode illustrated in FIG. 13.

When the earphone hook 10C is worn with the first wearing mode illustrated in FIG. 12, the hook portion 51 is worn on the auricle 100 in a state in which the cable-holding portion 202 is first inserted into the insertion portion 61. Then, the speaker housing 203 is set in the external auditory meatus. In this way, the earphone 200 is worn on the auricle 100 using the earphone hook 10C in a state in which the cable 201 directly extends downward from the insertion portion 61.

When the earphone hook IOC is worn with the second wearing mode illustrated in FIG. 13, the cable 201 is accommodated in the cable guide groove 62 after the earphone hook 10C is worn with the first wearing mode. In this way, the earphone 200 is worn on the auricle 100 using the earphone hook 10C in a state in which the cable 201 is wrapped rearward around the auricle 100.

The technical scope of the present invention is not limited to the above-described embodiments, and various modifications can be made without departing from the gist of the present invention.

For example, although the GB-2 point P1 and the GB-12 point P2 are stimulated by the magnetic stimulation portions 18 and 19 in the above-described embodiment, embodiments are not limited thereto. Acupuncture points other than the GB-2 point P1 and the GB-12 point P2 may be stimulated as long as it is an acupuncture point located around the auricle 100.

Although the constitution in which two magnetic stimulation portions 18 and 19 are provided is described in the above-described embodiment, embodiments are not limited thereto, and the number of magnetic stimulation portions 18 and 19 may be appropriately changed.

Although an earphone hook to which the earphone 200 is detachably attached is described as an example of a beneficial body instrument of the present invention in the above-described embodiment, embodiments are not limited thereto. For example, the present invention may be applied to anything that can be worn on the auricle 100 such as headphones, eyeglasses, masks, and accessories.

Further, in addition to the above-described materials, the magnetic stimulation portions 18 and 19 may contain germanium, magnesium, titanium, hematite, tourmaline, or the like. By containing germanium, magnesium, or titanium in the magnetic stimulation portions 18 and 19, blood flow can be further promoted by negative ions generated from the materials. Also, by containing hematite in the magnetic stimulation portions 18 and 19, blood flow can be further promoted. Further, by containing tourmaline in the magnetic stimulation portions 18 and 19, blood flow can be further promoted by negative ions or heat generated from tourmaline and minerals eluted from tourmaline.

In addition, elements in the above-described embodiments may be appropriately substituted by known elements without departing from the gist of the present invention.

### INDUSTRIAL APPLICABILITY

According to the above-described beneficial body instrument, beneficial effects can be easily obtained by stimulating acupuncture points around an auricle.

### REFERENCE SIGNS LIST

10A, 10B, 10C Earphone hook (beneficial body instrument)

11 Hook portion

13, 60 Earphone-holding portion

18 First magnetic stimulation portion

19 Second magnetic stimulation portion

38, 39, 52, 53 Protrusion

100 Auricle

200 Earphone

1. A beneficial body instrument, comprising:
a hook portion detachably worn on an auricle to be wrapped around the auricle; and
magnetic stimulation portions provided at the hook portion to stimulate acupuncture points around the auricle.

2. The beneficial body instrument according to claim 1, wherein the magnetic stimulation portions include
a first magnetic stimulation portion provided at a portion of the hook portion located in front of the auricle to stimulate a Gall Bladder 2 (GB-2) acupuncture point; and
a second magnetic stimulation portion provided at a portion of the hook portion located behind the auricle to stimulate a Gall Bladder 12 (GB-12) acupuncture point.

3. The beneficial body instrument, according to claim 1, wherein:
at the portions of the hook portion at which the magnetic stimulation portions are provided, protrusions protruding toward a user in a state in which the hook portion is worn on the auricle are provided.

4. The beneficial body instrument, according to claim 1, wherein:
the hook portion is configured to be deformable.

5. The beneficial body instrument, according to claim 1, wherein:
an earphone-holding portion configured to detachably hold an earphone is provided at the hook portion.
